(19) United States
(12) Patent Application Publication
(10) Pub. No.: US 2014/0343473 A1
(43) Pub. Date: Nov. 20, 2014

(54) CARPAL TUNNEL GLOVE

(71) Applicant: NEW HAMPTON CREATIONS, INC., New York, NY (US)

(72) Inventor: David Hoffman, Wantagh, NY (US)

(73) Assignee: NEW HAMPTON CREATIONS, INC., New York, NY (US)

(21) Appl. No.: 13/895,833

(22) Filed: May 16, 2013

(51) Int. Cl. A61F 5/01

(52) U.S. Cl. CPC A61F 5/013
USPC 602/21

(57) ABSTRACT

A glove is for the treatment and/or prevention of repetitive stress injury such as carpal tunnel syndrome includes an exterior fabric portion fabricated from synthetic polymeric material, and an interior fabric portion fabricated from pure metallic silver yarn joined to the exterior fabric.

### BACKGROUND

1. Field of the Invention

The present invention relates to an orthopedic device and particularly to a glove for the prevention or treatment of carpal tunnel syndrome.

2. Background of the Art

Various glove devices have been made for the prevention or treatment of carpal tunnel syndrome. For example, U.S. Pat. No. 5,376,066 to Phillips et al. discloses a sleeve formed of an elastic material such as neoprene which stretches longitudinally and transversely. A thumb receiving opening is provided in the intermediate portion of the sleeve. The proximal end of the sleeve includes a releasably securable strap located adjacent the wrist of the person for wrapping about a portion of the wrist to secure the glove-like device in place. When the device is in place the person's fingers extend through the respective finger openings and the person's thumb extends through the thumb opening. The dorsal side of the sleeve is biased backward for extending the person's hand in flexion at a slight angle, e.g., 25-30 degrees, to the wrist axis so that it is in a neutral position. The intermediate portion of the sleeve is configured to tightly encircle the palm and contiguous dorsal portion of the hand to resist the palm from spreading. A pad may be provided on the palmar side of the glove-like member at the interface of the intermediate portion and proximal end portion.

U.S. Pat. No. 5,405,357 to Rowe-Lanzisera et al. discloses an acupressure glove for application of self-stimulation of acupressure points on the hand and wrist. The glove is intended for carpal tunnel syndrome as well as other ailments.

U.S. Pat. No. 4,850,341 discloses a glove for preventing or inhibiting carpal tunnel syndrome. The glove has a pad disposed therein for protecting the median nerve of a wearer's hand. In an embodiment the pad is secured to the glove body and extends from near the wrist opening of the glove to about the center of the portion of the glove which covers the palm. The pad is made of a resilient flexible material such as foam rubber to provide protection from vibrations and shocks.

Prior known gloves are typically stiff and do not allow free movement of the hand. What is needed is a glove which stimulates blood flow to the region affected by carpal tunnel syndrome and which is sufficiently flexible to allow free movement of the hand.

### SUMMARY

A glove is provided herein for the treatment and/or prevention of repetitive stress injury such as carpal tunnel syndrome. The glove comprises an exterior fabric portion fabricated from synthetic polymeric material, and an interior fabric portion fabricated from 100% pure silver yarn joined to the exterior fabric.

Unlike previous carpal tunnel gloves, the glove of the invention is soft and completely movable, and it can be worn all day without discomfort. Moreover the glove of the invention is sufficiently stretchable to conform to the shape of the hand and wrist.

### BRIEF DESCRIPTION OF THE DRAWINGS

Various embodiments are described below with reference to the drawings wherein:

FIG. 1 illustrates the use of the glove of the invention on a human hand; and

FIG. 2 is a cross-sectional view of the glove.

### DETAILED DESCRIPTION OF PREFERRED EMBODIMENT(S)

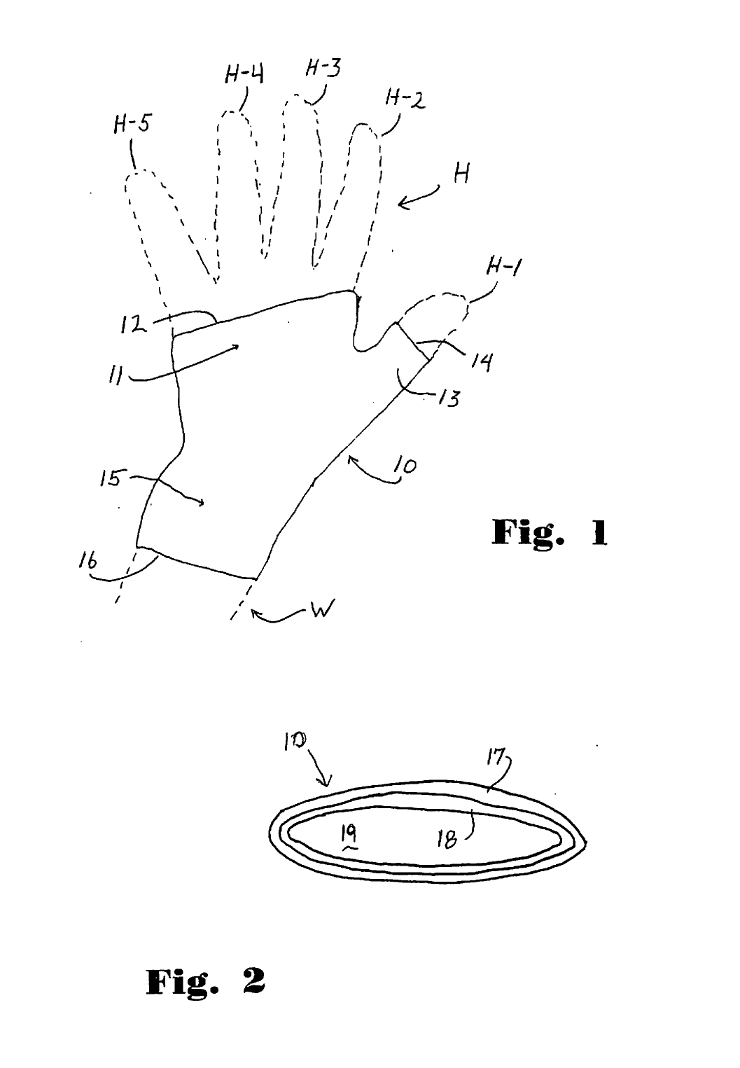

Referring now to FIGS. 1 and 2, glove 10 of the invention is a unitary article and includes a palm portion 11, a thumb portion 13 and a wrist portion 15. Palm portion 11 includes an opening 12 configured and dimensions to allow four fingers of the human hand, i.e., forefinger H-2, middle finger H-3, ring finger H-4 and little finger H-5, to extend therethrough. Thumb portion 13 extends from palm portion 11 and includes an opening 14 configured and dimensioned to allow the thumb H-1 to extend therethrough. Wrist portion 15 includes an opening 16 to accommodate the wrist W of the human hand.

Referring especially now to FIG. 2, the glove 10 comprises a dual layer fabric defining an interior space 19. Outer layer 17 can be fabricated from a synthetic fiber such as nylon, acrylic, polyolefin or polyester. In a preferred embodiment, outer layer 17 is fabricated from a dyeable ring spun polyester yarn. Such yarn is known and commercially available. In contrast to other types of polyester which can be abrasive to the feel, ring spun polyester is soft and flexible and can be worn comfortably throughout the day. The inner layer 18 is fabricated from a 100% metallic silver yarn. Silver yarns suitable for use in the invention are commercially available. In an embodiment the outer layer 17 preferably has a thickness of about 3 mm, and the inner silver layer 18 preferably has a thickness of about 1 mm. The overall thickness of the glove is preferably about 4 mm.

The dual layer fabric is stretchable and flexible so as to conform to the shape of the hand. Typically, the length of the glove 10 (i.e., the distance between opening 12 and opening 16) ranges from 6 inches to 10 inches to provide sizes of small, medium, large and extra large. The glove 10 can be made on a sock machine. Sock machines can have various numbers of needles, for example from 40 to over 200. In a preferred embodiment the glove of the invention is fabricated on a 168 needle sock machine, although other numbers of needles can be employed when appropriate. It should be noted that shear knitting machines do not provide a product with the softness and flexibility of the present invention.

The inner liner 18 of pure metallic silver yarn when in contact with the skin draws blood flow to the area of contact tending to reduce pain. Moreover, the silver has antimicrobial properties. The glove 10 provides full mobility of the hand and can fit underneath shirt sleeves. The polyester layer 17 can be dyed to any desired color or pattern and can include on its outer surface insignia, text, logos or other indicia.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. A glove comprising:
an exterior fabric portion fabricated from synthetic polymeric material, and
an interior fabric portion fabricated from pure metallic silver yarn joined to said exterior fabric.

2. The glove of claim 1 wherein the exterior fabric is ring a spun polyester yarn

3. The glove of claim 2 wherein said polyester yarn is dyed.

4. The glove of claim 1 wherein said glove is a unitary article having a palm portion, a wrist portion, an opening in the palm portion configured to allow four fingers of a human hand to pass therethrough, an opening in the wrist portion to accommodate a human wrist, a thumb portion, and an opening in the thumb portion to accommodate a human thumb.

5. The glove of claim 1 wherein said exterior fabric is stretchable.

6. The glove of claim 1, wherein the glove has a length of from about 6 to about 10 inches.

7. The glove of claim 1 wherein an outer surface of the exterior fabric includes indicia.

8. The glove of claim 1 wherein the exterior fabric portion has a thickness of about 3 mm.

9. The glove of claim 8 wherein the interior fabric portion has a thickness of about 1 mm and the overall thickness of the glove is about 4 mm.

10. The glove of claim 1 fabricated on a sock machine.

11. The glove of claim 10 wherein the sock machine is a 168 needle sock machine.

12. A glove comprising:
an exterior fabric portion fabricated from a ring spun polyester yarn, and
an interior fabric portion fabricated from pure metallic silver yarn joined to said exterior fabric, wherein said glove is a unitary article having a palm portion, a wrist portion, an opening in the palm portion configured to allow four fingers of a human hand to pass therethrough, an opening in the wrist portion to accommodate a human wrist, a thumb portion, and an opening in the thumb portion to accommodate a human thumb.

13. The glove of claim 12 wherein said exterior fabric is stretchable.

14. The glove of claim 12, wherein the glove has a length of from about 6 to about 10 inches.

15. The glove of claim 12 wherein an outer surface of the exterior fabric includes indicia.

16. The glove of claim 12 wherein the exterior fabric portion has a thickness of 1 about 3 mm.

17. The glove of claim 16 wherein the interior fabric portion has a thickness of about 1 mm, and the overall thickness of the glove is about 4 mm.

18. The glove of claim 12 fabricated on a sock machine.

19. The glove of claim 18 wherein the sock machine is a 168 needle sock machine.

20. The glove of claim 12 wherein the exterior fabric portion has an outer surface including indicia.
